(19) United States
(12) Patent Application Publication
(10) Pub. No.: US 2026/0283611 A1
(43) Pub. Date: Sep. 24, 2026

(54) Suture Tensioning Anchor Inserter

(71) Applicant: Arthrex, Inc., Naples, FL (US)

(72) Inventors: Thomas DOONEY, JR., Naples, FL (US); Sam AKHAVAN, Naples, FL (US); Stephan M. BOSCH, Naples, FL (US); John M. TOKISH, Scottsdale, AZ (US)

(73) Assignee: Arthrex, Inc., Naples, FL (US)

(21) Appl. No.: 19/557,422

(22) Filed: Mar. 5, 2026

Related U.S. Application Data
(60) Provisional application No. 63/774,774, filed on Mar. 20, 2025.

(51) Int. Cl. A61B 17/04

(52) U.S. Cl. CPC A61B 17/0401; A61B 2017/0409; A61B 2017/0445; A61B 2017/0453

(57) ABSTRACT

Surgical constructs, assemblies, kits and methods of tissue fixation are disclosed. A suture tensioning anchor inserter is provided with a tensioning attachment that allows tensioning of suture directly on the inserter. The tensioning attachment may include a pair of paddles that can twist in one direction to add tension to the sutures in a repair. The paddles eliminate suture tensioning by hand, providing a mechanical advantage to add additional suture tension.

### CROSS-REFERENCE TO RELATED APPLICATIONS

This application claims priority to U.S. Provisional Patent Application No. 63/774,774 filed Mar. 20, 2025, the disclosure of which is hereby incorporated by reference in its entirety herein.

### BACKGROUND

The present disclosure relates to the field of surgery and, more particularly, to surgical instruments and methods of surgical repairs.

### SUMMARY

Surgical instruments, assemblies, and methods are disclosed. A surgical instrument is a suture anchor inserter provided with an attachment that allows tensioning of sutures directly on the inserter. In some implementations, the attachment may include a pair of paddles that can twist in one direction to add tension to the sutures in a repair. The paddles eliminate suture tensioning by hand providing a mechanical advantage to add additional suture tension. The paddles also lock in the suture tension as the paddles can only twist in one direction, so the suture tension will not loosen. In some implementations, the suture anchor may be a swivel anchor. In some implementations, the suture anchor may be a push-in anchor. In some implementations, a swivel anchor may be locked and secured within tissue by inserting a cannulated fixation device to contact an anchor eyelet.

A surgical assembly may include an inserter and an anchor for knotless tissue fixation. An anchor may be employed with an inserter provided with a tensioning mechanism that allows sutures from an anchor eyelet to engage the tensioning mechanism. A tensioning mechanism may include a plurality of paddles configured to ratchet and twist in one direction. In some implementations, the anchor may be a swivel anchor. In some implementations, a swivel anchor may be locked and secured within tissue by inserting a cannulated fixation device to contact the eyelet.

Methods of surgeries are also disclosed. Surgical repairs may be conducted with an inserter designed to tension suture attached to an eyelet of a suture anchor that is secured into bone with the inserter. Sutures attached to a suture eyelet that is in or within the proximity of a bone hole are cleated into rotating ends of each paddle. The paddle ends are rotated to independently and evenly add tension to the sutures in the repair. In this manner, the tension is locked in and cannot loosen. The anchor may be inserted into a prepared bone hole locking in the suture tension. The sutures may be removed from the paddles and cut off to finalize the repair.

These and other features and advantages of this disclosure will become apparent and will be understood from the following detailed description of the various aspects of the disclosure taken in conjunction with the accompanying drawings.

### BRIEF DESCRIPTION OF THE DRAWINGS

FIG. 1 illustrates a schematic view of an anchor assembly.

FIGS. 2-4 illustrate subsequent view of a method of tissue repair with the anchor assembly of FIG. 1.

FIG. 5 illustrates a schematic view of another anchor assembly.

FIGS. 6 and 7 illustrate enlarged views of the paddle of the anchor assembly of FIG. 5.

### DETAILED DESCRIPTION

Assemblies, apparatus and methods for tissue fixation using suture anchors are disclosed. In some implementations, a swivel anchor may be used with an inserter tensioner configured to securely engage and retain an eyelet carrying one or more flexible strands and to also evenly tension ends of the one or more flexible strands. A tensioning mechanism of the inserter tensioner allows ends of the flexible strands passed through the eyelet to be securely attached to the tensioning mechanism and to be evenly tensioned. In some implementations, the tensioning mechanism includes a pair of paddles provided with one or more cleats that engage and secure the ends of the flexible strands. Rotation of the paddles allows tensioning of the flexible strands.

In some implementations, the shaft of the inserter tensioner is provided with a tip that mates with the suture eyelet. At least one flexible strand is passed through the eyelet on the inserter tensioner. The inserter tensioner may be also used to engage and lock the eyelet in a hole in bone by inserting a cannulated fixation device (for example, an anchor, an interference plug or screw, or an implant) to contact the eyelet.

A surgical assembly may include a suture anchor for knotless or knotted tissue fixation. In some implementations, the anchor may be a swivel anchor. In some implementations, the anchor may be a knotless swivel anchor. In some implementations, a swivel anchor may be employed with an inserter tensioner provided with a tensioning mechanism that tensions one or more sutures passed through a suture eyelet. A tensioning mechanism may include a pair of paddles configured to rotate in same or different directions to allow tensioning of the sutures passed through the suture eyelet. The tensioning mechanism may allow even tensioning of the sutures on each side of the inserter tensioner. In some implementations, the inserter tensioner may be solid and the flexible strand(s) such as suture(s) extend along an outer surface of the inserter tensioner and along its longitudinal sides. A swivel anchor may be locked and secured within tissue by inserting a cannulated fixation device (for example, an anchor, an interference plug or screw, or an implant) to abut and contact the suture eyelet.

Methods of tissue repairs are also disclosed. A method for knotless fixation of anatomical tissue with a knotless anchor may include: (i) capturing at least one flexible strand attached to a first tissue to be secured to a second tissue with an anchor eyelet secured to an anchor inserter; (ii) securing the anchor eyelet with the at least one flexible strand into the second tissue; (iii) subsequently, securing ends of the at least one flexible strand to a tensioning mechanism of the anchor inserter and tensioning the at least one flexible strand to approximate the first tissue to the second tissue; and (iv) securing a fixation device (anchor) over or in contact with the anchor eyelet. The tensioning mechanism may include a plurality of rotating paddles. The rotating paddles may turn in one direction. The rotating paddles may ratchet. The knotless anchor may be a swivel anchor. The knotless anchor may be a push-in anchor.

The anchor eyelet may be secured into the second tissue by providing a cannulated fixation device (for example, an anchor, an interference plug or screw, or an implant) to abut and contact the anchor eyelet. The anchor eyelet and cannulated fixation device may form and be part of a swivel anchor. The eyelet may be a swivel anchor implant that allows secure engagement and retention of the cannulated fixation device. The first tissue may be different from the second tissue. In some implementations, the first tissue may be soft tissue and the second tissue may be bone.

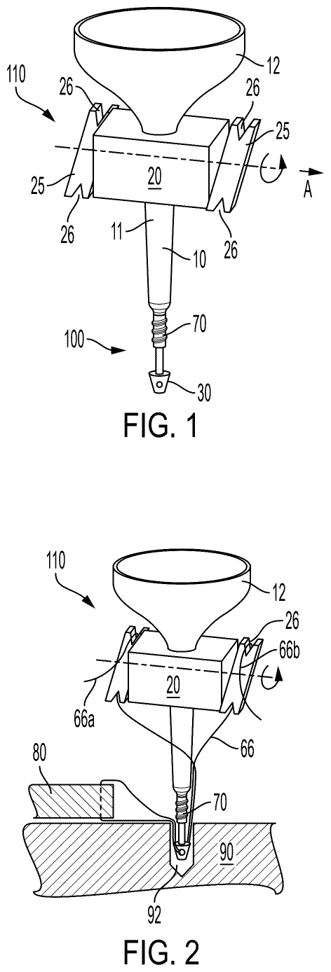

Referring now to the drawings, where like elements are designated by like reference numerals, FIGS. 1-7 illustrate various structural elements of surgical assembly 110, 110a with inserter tensioner 10, 10a and anchor 100. Inserter tensioner 10, 10a may be a suture anchor inserter tensioner; a tensioning inserter; a driver tensioner; a driver; a suture tensioning anchor inserter; or a SwiveLock® anchor inserter tensioner. Anchor 100 may be a swivel anchor; a suture anchor; an implantable device; an implant; an anchor assembly; an anchor system; a tensionable, knotless construct; or a SwiveLock® anchor. FIGS. 2-4 illustrate steps of an exemplary repair 101 with surgical assembly 110 and inserter tensioner 10.

Reference is now made to FIG. 1, which illustrates details of assembly 110 with inserter tensioner 10 and anchor 100. Inserter tensioner 10 may be a solid, cannulated driver including shaft 11 and an inserter handle 12. A pair of paddles 25 are provided at a most proximal end of driver 10 on each side of thumb pad 20. In some implementations, the paddles may be tabs or protuberances designed to securely engage ends of one or more flexible strands 66 passed through suture anchor eyelet 30 of anchor 100, and as detailed below.

In some implementations, and as illustrated in FIG. 1, two paddles 25 are provided symmetrically positioned relative to the longitudinal axis of shaft 11. Paddles 25 may extend about parallel to longitudinal axis of shaft 11 and may have similar configurations and dimensions (as shown in FIG. 1) or may have different configurations and dimensions. In some implementations, paddles 25 are configured to rotate in direction A and are provided with a ratchet mechanism. Paddles 25 may rotate simultaneously or independently from each other. In some implementation, each paddle 25 may be provided with at least one cleat, slot, opening or channel 26 to allow secure engagement of various portions or lengths of flexible strand 66. Preferably, the cleats, slots, openings or channels 26 may have a shape and geometry that is complementary to that of flexible strand 66 to allow secure engagement and retainment of the at least one flexible strand 66. In some embodiments, each paddle 25 is provided with two symmetrical cleats 26, as shown in FIGS. 1-4.

Eyelet 30 of anchor 100 is detachable from inserter tensioner 10 and provided with a body that includes a distal tip with a transverse through-hole or suture opening (suture eyelet) to allow passage of at least one flexible strand (suture) through it. In some implementations, eyelet 30 may be provided with a ribbed surface or an uneven surface, to allow better engagement of bone during anchor eyelet insertion within a bone hole or opening. Eyelet 30 may be a suture eyelet; a suture anchor eyelet; an anchor eyelet; an implant; a swivel implant; or a swivel anchor implant.

Reference is now made to FIGS. 2-4, which illustrate exemplary repair 101 with surgical assembly 110 and inserter tensioner 10.

FIG. 2: At least one flexible strand 66 (suture 66) is passed through a first tissue 80 that is intended to be repaired and that is approximated to a second tissue 90. Insert eyelet 30 with captured flexible strand 66 within hole or opening 92 formed within second tissue 90. In some implementations, first tissue 80 may be soft tissue and second tissue 90 may be bone. In some implementations, first tissue 80 may be soft tissue or bone. Eyelet 30 of the anchor 100 can either be in or within the proximity of prepare bone hole 92. Ends 66a, 66b of suture 66 are then cleated onto cleats 26 of paddles 25.

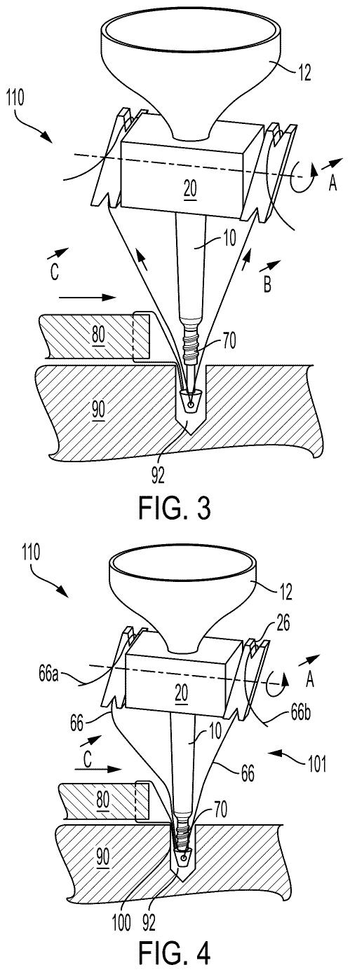

FIG. 3: The paddle ends are rotated, independently adding tension to the sutures 66 in the repair. The tension is locked in and cannot loosen. The repair is tensioned by pulling first tissue 80 in the direction of arrow C to approximate first tissue 80 to second tissue 90.

FIG. 4: Subsequent to the tensioning, anchor 70 (fixation device or cannulated fixation device 70) can be inserted into the prepared bone hole 92 locking in the suture tension. The sutures 66 can be removed from the paddles 25 and cut to finalize repair 101. FIG. 4 depicts the entire construct once fully inserted. Anchor 100 may be a swivel anchor 100. Anchor 100 includes eyelet 30 and cannulated fixation device 70 (anchor 70). At least one flexible strand 66 passes through a suture opening of anchor eyelet 30, extending on an outer surface of driver shaft 11.

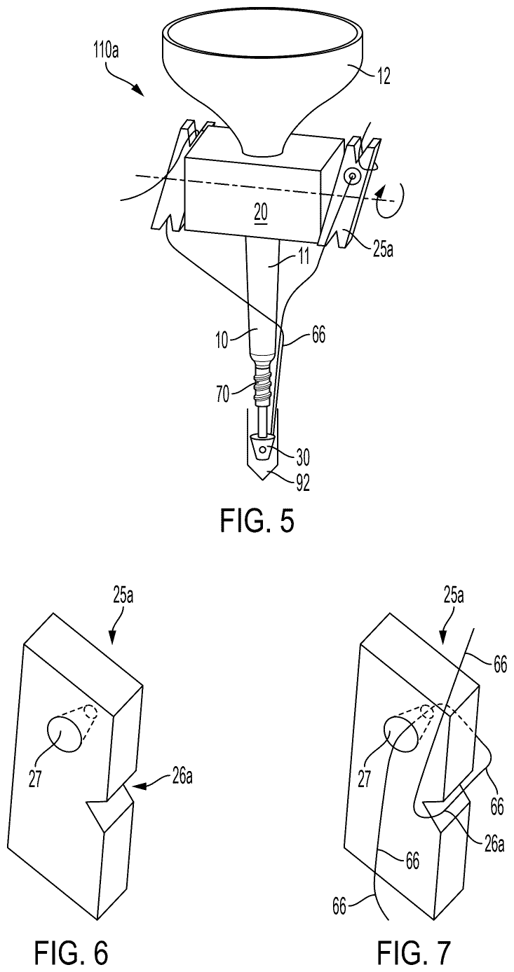

Reference is now made to FIG. 5 which illustrates another exemplary assembly 110a which is about similar to assembly 110 detailed above but differs in the configuration of paddles 25a. As illustrated, at least one of the paddles 25a can have the configuration shown in FIGS. 6 and 7.

As shown in FIGS. 5 and 7, the flexible strand 66 (suture 66) can be fixated on paddle 25a using a tapered through hole 27 and a side cleat 26a. This configuration makes it easier to load and release the suture 66 after it has been tightened. It also forces the user to load the paddle that will complement the (one-way) direct of spin of the paddle.

In some implementations, the fixation device 70 may be advanced into the bone socket by holding thumb pad 20 as the driver/inserter handle is turned clockwise. When the fixation device 70 is fully seated, the most distal end of the fixation device 70 abuts and contacts the most proximal end of eyelet 30 to optimize the stability of the swivel anchor construct 100. As previously stated, the swivel anchor construct 100 is composed of eyelet 30 and fixation device 70.

In some implementations, anchor construct 100 may be a swivel anchor about similar to a two-piece construct such as a SwiveLock® anchor. A two-piece construct is an Arthrex SwiveLock® anchor, disclosed in U.S. Pat. No. 8,012,174 and U.S. Pat. No. 9,005,246, the disclosures of both of which are fully incorporated by reference in their entirety herein. Although the anchor 100 has been described above as including anchor eyelet 30 having a suture opening or hole to allow passage of one or more flexible strands 66 through it, it must be understood that the disclosure is not limited to this exemplary-only embodiment and contemplates eyelet 30 having other various geometries, such as, for example, a forked tip for capturing one or more flexible strands 66, as detailed in, for example, U.S. Pat. No. 10,045,770, the disclosure of which is also fully incorporated by reference in its entirety herein.

In some implementations, surgical assembly 110, 110a may comprise a tensioning inserter 10 with a cannulated driver assembly having a distal end and a rod passing slidably and rotatably through the driver assembly and a tensioning mechanism 25, 25a; a swivel anchor implant 30, wherein a distal tip of the rod is configured to releasably accept at least part of the swivel anchor implant 30 therein; a fixation device 70 configured to be assembled onto the rod, wherein the distal end of the driver assembly is engageable with the fixation device 70; and at least one suture 66 or suture tape 66, wherein the swivel anchor implant 30 is configured to capture the suture or suture tape 66 for implantation into a hole 92, wherein the swivel anchor implant 30 and the fixation device 70 are configured to be held in place axially in the hole 92 using the tensioning inserter 10, with the fixation device 70 assembled on the rod and the swivel anchor implant 30 at the distal tip of the rod, and wherein the fixation device 70 is rotatable by the tensioning inserter to advance the fixation device 70 axially into the hole 92, with the swivel anchor implant 30 disposed distal to the fixation device 70 in the hole 92.

In some implementations, the fixation device 70 is a cannulated screw. The driver assembly may include a handle opposite the distal end. The fixation device 70 may be movable axially relative to the rod and configured to engage and to be rotationally fixed relative to a portion of the tensioning inserter. The swivel anchor implant 30 may be provided with a closed aperture for capturing the suture 66 or suture tape 66. The swivel anchor implant 30 may be provided with a forked tip for capturing the suture 66 or suture tape 66.

In some implementations, a method of tissue fixation 101 may comprise securing a first portion of a suture 66 to a first tissue 80 to be fixated; and anchoring a second portion of the suture 66 into an opening or socket 92 of a second tissue 90 using a swivel implant 30, a separate cannulated fixation device 70 having a threaded body, the swivel implant 30 being attached onto a tip of a driver assembly, the driver assembly comprising a cannulated driver with a shaft, a tensioning inserter 10 with a handle having a tensioning mechanism 25, 25a and a tube that passes slidably and rotatably through the cannulated driver, the tube having a tip that accepts the swivel implant 30, the swivel implant 30 being engaged with the tip of the tube, the tube further having an outer surface for receiving the cannulated fixation device 70, the driver assembly being pre-loaded with the cannulated fixation device 70, the swivel implant 30 swivels relative to the cannulated fixation device 70, the swivel implant 30 comprising a tip and an anchor body, wherein the tip of the swivel implant is configured to capture the suture 66 attached to the first tissue 80, wherein the suture 66 is anchored into the opening or socket 92 by the steps of:
capturing the suture 66 attached to the tissue 80 with the swivel implant 30; inserting the swivel implant 30 with the captured suture 66 into the opening or socket 92 with the tensioning inserter 10;
securing ends 66a, 66b of the captured suture 66 to cleats 26, 26a and tapered hole 27 of paddles 25, 25a of the tensioning mechanism of the tensioning inserter 10 and, subsequently, tensioning the captured suture 66 to approximate the first tissue 80 to be fixated to the second tissue 90; and
advancing the cannulated fixation device 70 using the driver assembly into the opening or socket 92 so that the cannulated fixation device 70 engages the opening or socket 92, thereby securing the suture 66 and providing tissue fixation without tying any knots in the suture 66. The swivel implant 30 may include a closed aperture for capturing suture 66.

Flexible strand 66 may be passed through the tissue and can be secured either using a single anchor (such as anchor construct 100) or a plurality of anchors. In addition, various anchors, such as those noted above and others, may be used interchangeably with only slight variations in the procedure. In some implementations, a plurality of flexible strands 66 may be passed through the suture eyelet of eyelet 30.

An inserter 10 can be a knotless anchor tensioning inserter 10 provided with a tensioning mechanism. In some implementations, a tensioning mechanism can include one or more paddles 25, 25a that allow even tensioning directly onto the inserter instrument. A surgical inserter 10 can provide bone anchor insertion and suture/repair tensioning with increased suture tensioning at the repair site. The anchor 100 may be a swivel anchor 100.

A surgical assembly 110, 110a may include an anchor 100 for knotless tissue fixation. An anchor 100 may be employed with a tensioning inserter 10 provided with a tensioning mechanism 25, 25a that allows engagement and tensioning of ends 66a, 66b of at least one flexible strand 66 passed through (attached to) an eyelet 30. A tensioning mechanism 25, 25a may include one or more paddles 25, 25a provided on the tensioning inserter 10 that include cleats 26, 26a and/or tapered through-hole 27 designed to retain the ends of the flexible strand 66. Rotation of the paddles 25, 25a allows tensioning of the flexible strand 66 prior to anchor fixation into bone 90. In some implementations, the anchor 100 may be a swivel anchor 100. In some implementations, a swivel anchor 100 may be locked and secured within tissue by inserting a cannulated fixation device 70 to contact suture anchor eyelet 30.

The disclosure also provides an implant fixation device that tensions a flexible strand (for example, a suture) with a fixation device (either swivel-in or push-in) in between two separate points of tension (such as paddle 25 and bone hole 92). In this manner, suture tensioning occurs subsequently to the suture insertion into bone hole (subsequent to inserting the eyelet with suture into bone hole 92); suture insertion does not occur after its tensioning.

Methods of tissue repairs 101 are also disclosed. A method for knotless fixation of anatomical tissue with a knotless anchor 100 may include: (i) capturing at least one flexible strand 66 attached to first tissue 80 with an anchor eyelet 30 secured to a suture tensioning anchor inserter 10; (ii) securing the anchor eyelet 30 with the at least one flexible strand 66 into second tissue 90; (iii) securing ends 66a, 66b of the at least one flexible strand 66 to a tensioning mechanism 25, 25a of the suture tensioning anchor inserter 10 and tensioning the at least one flexible strand 66 to approximate first tissue 80 to second tissue 90; and (iv) subsequently, securing a fixation device 70 (anchor) over or in contact with the anchor eyelet 30. The tensioning mechanism 25 may include a plurality of rotating paddles 25, 25a. The rotating paddles 25, 25a may turn in one direction. The rotating paddles 25, 25a may ratchet. The rotating paddles 25a may be provided with a side cleat 26a and a through hole 27. The through hole 27 may be a tapered through hole. The knotless anchor 100 may be a knotless swivel anchor. The knotless anchor 100 may be a push-in anchor.

A method for fixation of anatomical tissue during surgical applications may include employing an anchor assembly 110, 110a that includes a suture anchor inserter tensioner 10 having a tensioning mechanism 25, 25a. The method may include: (i) capturing, with an anchor eyelet 30, at least one flexible strand 66 passed through or around first tissue 80 to be positioned relative to a second tissue 90; (ii) attaching a tip of the inserter tensioner 10 to the anchor eyelet 30; (iii) inserting the anchor eyelet 30 into a hole 92 formed with the second tissue 90; (iv) attaching ends 66a, 66b of the at least one flexible strand 66 to the tensioning mechanism 25, 25a and tensioning the flexible strand 66 to approximate the first tissue 80 to the second tissue 90; and (vi) advancing a fixation device 70 over and in contact with the anchor eyelet 30, to secure the anchor eyelet 30 with the captured flexible strand 66 into the second tissue 90. Ends 66a, 66b of the at least one flexible strand 66 may be passed through a tapered through hole 27 and secured to a side cleat 26a of at least one paddle 25a. The anchor eyelet 30 may be secured into the second tissue 90 by providing the fixation device 70 (for example, a cannulated fixation device, an anchor, an interference plug or screw, or an implant) to abut and contact the anchor eyelet 30. The anchor eyelet 30 and cannulated fixation device 70 may form and be part of a swivel anchor 100. The eyelet 30 may be an anchor eyelet 30 that allows secure engagement and retention of the cannulated fixation device. The first tissue may be different from the second tissue. In some implementations, the first tissue may be soft tissue and the second tissue may be bone.

In some implementations, anchor 100 detailed above may be employed with additional independent fixation devices such as, for example, knotted anchors, knotless anchors, or all-suture anchors, or any devices that confer secure attachment and fixation of first tissue to a second tissue. Additional fixation devices that may be used with anchor 100 and assembly 110, 110a are, for example, knotless anchors such as a two-piece Arthrex PushLock® anchor, disclosed in U.S. Pat. No. 7,329,272, or Arthrex SwiveLock® anchors, disclosed in U.S. Pat. No. 8,012,174 and U.S. Pat. No. 9,005,246, the disclosures of both of which are fully incorporated by reference in their entirety herein.

The constructs, systems, and assemblies of the present disclosure may be employed in various soft tissue repairs and fixations, for example, fixation of soft tissue to bone and for any tissue positioning and/or tissue adjustment applications. The suture tensioning anchor inserter, assemblies and anchors of the present disclosure may be utilized in surgical procedures such as rotator cuff repair, Achilles tendon repair, patellar tendon repair, ACL/PCL reconstruction, hip and shoulder reconstruction procedures, AC joint reconstruction, syndesmosis reconstruction, quad/patellar tendon rupture repair, hallux-valgus repair, proximal and/or distal biceps tendon repair, humerus and radius repair, and any other tendon repair to bone, among many others. These repairs may be conducted in a knotless manner.

Flexible strand 66 may be any flexible coupler and may be formed of various flexible materials and strands such as round suture, flat suture, ribbon, or flat tape (for example, suture tape) or combination of suture and tape. Exemplary materials may include suture, silk, cotton, nylon, polypropylene, polyethylene, ultrahigh molecular weight polyethylene (UHMWPE), polyethylene terephthalate (PET), and polyesters and copolymers thereof, or combinations thereof. Flexible strand 66 may have cross-sections of various forms and geometries, including round, oval, rectangular, or flat, among others, or combination of such forms and geometries. In some implementations, flexible strand 66 may be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided with at least one other fiber, natural or synthetic, to form lengths of suture material. Flexible strand 66 may be braided or multi-filament suture such as FiberTape® suture tape (as disclosed in U.S. Pat. No. 7,892,256, the disclosure of which is incorporated in its entirety herewith). Flexible strand 66 may include elastic material. Flexible strand 66 may consist essentially of elastic suture.

Various regions or sections of flexible strand 66 may be coated and/or provided in different colors for easy manipulation during the surgical procedures. Easy identification of suture in situ is advantageous in surgical procedures, particularly during arthroscopic surgeries, endoscopic and laparoscopic procedures.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A method of tissue fixation comprising:
capturing at least one flexible strand attached to a first tissue with a swivel implant of a tensioning inserter;
inserting the swivel implant with the at least one flexible strand into an opening or socket into a second tissue;
securing ends of the at least one flexible strand to a tensioning mechanism of the tensioning inserter, and tensioning the at least one flexible strand to approximate the first tissue to the second tissue; and
advancing a cannulated fixation device into the opening or socket so that the cannulated fixation device engages the opening or socket, thereby securing the at least one flexible strand and providing knotless fixation of the first tissue to the second tissue.

2. The method of claim 1, wherein the tensioning mechanism includes a pair of paddles provided with cleats to secure the at least one flexible strand.

3. The method of claim 2, wherein the paddles are configured to rotate to tension ends of the at least one flexible strand.

4. The method of claim 2, wherein the paddles are configured to independently rotate.

5. The method of claim 2, wherein the paddles are provided with a ratchet.

6. The method of claim 2, wherein the paddles are provided on a side of a thumb pad of the tensioning inserter.

7. The method of claim 1, wherein the first tissue is soft tissue and the second tissue is bone.

8. The method of claim 7, wherein the soft tissue is tendon, ligament, or graft.

9. The method of claim 1, wherein the swivel implant includes at least one eyelet to allow the at least one flexible strand to slidingly and removably pass therethrough.

10. The method of claim 1, wherein the tissue fixation is rotator cuff reconstruction.

11. The method of claim 1, wherein the at least one flexible strand is suture or suture tape.

12. The method of claim 1, wherein the at least one flexible strand includes an elastic.

13. A surgical instrument, comprising:
an inserter with a tensioner; and
a fixation device comprising a fixation portion and an eyelet extending distal of the fixation portion,
wherein the fixation portion is located between the tensioner and the eyelet.

14. The surgical instrument of claim 13, wherein the fixation portion is configured to translate distally along the inserter to fix the eyelet into a bone hole.

15. The surgical instrument of claim 13, wherein the tensioner includes two paddles.

16. The surgical instrument of claim 15, wherein each of the two paddles includes at least one cleat.

17. The surgical instrument of claim 15, wherein each of the two paddles includes a unidirectional mechanism.

18. The surgical instrument of claim 15, wherein each of the two paddles includes a ratchet.

19. The surgical instrument of claim 13, wherein the fixation device is a swivel anchor.

20. The surgical instrument of claim 13, wherein the fixation device is a push-in anchor.
